(19) United States
(12) Patent Application Publication
(10) Pub. No.: US 2021/0309079 A1
(43) Pub. Date: Oct. 7, 2021

(54) MOBILE BODY, SMELL CARTRIDGE, CONTROL DEVICE, AND PROGRAM

(71) Applicant: TOYOTA JIDOSHA KABUSHIKI KAISHA, Aichi-ken (JP)

(72) Inventor: Miyako SAKAI, Toyota-shi (JP)

(21) Appl. No.: 17/160,389

(22) Filed: Jan. 28, 2021

(30) Foreign Application Priority Data
Apr. 2, 2020 (JP) 2020-066932

(51) Int. Cl. B60H 3/00; A61L 9/12

(52) U.S. Cl. CPC B60H 3/0035; A61L 9/122

(57) ABSTRACT

A mobile body including: a smell releasing device that releases to the outside, a smell substance that generates smell; and a control device that controls the smell releasing device, in which when there is no release target that is predetermined as a target to which the smell substance is to be released within a predetermined range from the mobile body, the control device controls the smell releasing device to stop the smell substance from being released to the outside. This makes it possible to increase the time during which the smell substance can be released.

### CROSS-REFERENCE TO RELATED APPLICATION

This application claims priority to Japanese Patent Application No. 2020-066932 filed on Apr. 2, 2020, incorporated herein by reference in its entirety.

### BACKGROUND

### 1. Technical Field

The present disclosure relates to a mobile body, a smell cartridge, a control device, and a program.

### 2. Description of Related Art

Heretofore, as a mobile body, a vehicle that prohibits a window from being opened when the intensity of the internal smell is equal to or higher than a predetermined threshold value has been proposed (for example, see Japanese Unexamined Patent Application Publication No. 2019-127221 (JP 2019-127221 A)). In this mobile body, by prohibiting the window from being opened in this way, the internal smell is suppressed from being released to the outside.

### SUMMARY

In the above-mentioned mobile body, the smell substance is not expected to be released to the outside. As a method of actively releasing the smell substance to the outside, a method of constantly releasing the smell substance to the outside can be considered. However, since the amount of the smell substance that can be mounted on the mobile body is limited, if the smell substance is constantly released to the outside, the time during which the smell substance can be released will be shortened.

The main object of the mobile body, the smell cartridge, the control device, and the program of the present disclosure is to increase the time during which the smell substance can be released.

The mobile body, the smell cartridge, the control device, and the program of the present disclosure have taken the following measures to achieve the above-mentioned main object.

The overview of the mobile body is that the mobile body of the present disclosure includes:

a smell releasing device that releases to the outside, a smell substance that generates smell; and

a control device that controls the smell releasing device,

in which when there is a release target to which the smell substance is released within a predetermined range from the mobile body, the control device controls the smell releasing device to release the smell substance to the outside, and when there is no release target within the predetermined range from the mobile body, the control device controls the smell releasing device to stop the smell substance from being released to the outside.

In the mobile body of the present disclosure, when there is a release target to which a smell substance is released within a predetermined range from the mobile body, the smell releasing device is controlled to release the smell substance to the outside. As a result, the smell substance can be released to the release target. When there is no release target within the predetermined range from the mobile body, the smell releasing device is controlled to stop releasing the smell substance to the outside. When there is no release target within the predetermined range from the mobile body, it is not necessary to release the smell substance. Thus, by stopping the release of the smell substance to the outside, the consumption of the smell substance can be suppressed. As a result, the time during which the smell substance can be released can be increased. Here, the “predetermined range” is a range that is predetermined as a range in which the smell substance is desired to reach. Examples of the “release target” include humans and non-human animals.

In such a mobile body of the present disclosure, in a case in which there is the release target within the predetermined range from the mobile body, when a distance from the mobile body to the release target is long, the control device may control the smell releasing device to increase a reaching distance of the smell substance compared to when the distance is short. In this way, the smell substance can reach the release target more surely.

In this case, the smell releasing device has a storage device that stores the smell substance, a communication portion that allows communication between the storage device and the outside, a release blocking device that is able to block the smell substance from being released from the storage device to the communication portion, and a blower that takes in air and sends air to the communication portion, wherein when there is no release target within the predetermined range from the mobile body, the control device controls the release blocking device to block the smell substance from being released from the storage device to the communication portion, and when there is the release target within the predetermined range from the mobile body and the distance to the release target is long, the control device increases an air flow of the blower compared to when the distance is short. When there is no release target within the predetermined range from the mobile body, the release blocking device is controlled to block the smell substance from being released from the storage device to the communication portion. As a result, it is possible to stop the smell substance from being released to the outside by using the release blocking device. Further, when there is the release target within the predetermined range from the mobile body and the distance to the release target is long, the air flow of the blower is increased compared to when the distance is short. In this way, by increasing the air flow of the blower, the smell substance can reach the release target.

In a first mobile body of the present disclosure of an aspect in which the smell releasing device has a storage device, a communication portion, a release blocking device, and a blower, when there is a plurality of release targets within the predetermined range from the mobile body and a distance from the mobile body to the release target closest to the mobile body among the plurality of release targets is long, the control device may increase the air flow of the blower compared to when the distance is short. In this way, it is possible to suppress the inconvenience that a relatively large air flow hits the release target near the mobile body.

The mobile body of the present disclosure may include a direction adjusting device that adjusts a releasing direction of the smell substance, and when there is, on one surface side of the mobile body, a predetermined object in which release of the smell substance should be avoided, the control device may control the direction adjusting device to suppress the smell substance from being released to the one surface side. In this way, it is possible to suppress the release of the smell substance in the direction of the predetermined object.

The overview of the smell cartridge of the present disclosure is that the smell cartridge is used for a mobile body of an aspect of the present disclosure in which the smell releasing device has a storage device that stores a smell substance, that is, basically, the mobile body has the smell releasing device that releases the smell substance generating smell to the outside and a control device that controls the smell substance, in which when there is a release target to which the smell substance is released within a predetermined range from the mobile body, the control device controls the smell releasing device to release the smell substance to the outside, and when there is no release target within the predetermined range from the mobile body, the control device controls the smell releasing device to stop the smell substance from being released to the outside, in which the smell releasing device has the storage device that stores the smell substance, a communication portion that allows communication between the storage device and the outside, a release blocking device that is able to block the smell substance from being released from the storage device to the communication portion, and a blower that takes in air and sends air to the communication portion, in which when there is no release target within the predetermined range from the mobile body, the control device controls the release blocking device to block the smell substance from being released from the storage device to the communication portion, and when there is the release target within the predetermined range from the mobile body and the distance to the release target is long, the control device increases an air flow of the blower compared to when the distance is short, and in which the smell cartridge has a storage portion that functions as the storage device when used in the mobile body; and a control unit that functions as the control device when used in the mobile body.

The smell cartridge of the present disclosure includes: a storage portion that functions as the storage device when used in the mobile body; and a control unit that functions as the control device when used in the mobile body. In a mobile body using such a smell cartridge, when there is the release target to which the smell substance is released within the predetermined range from the mobile body, the smell releasing device is controlled to release the smell substance to the outside. As a result, the smell substance can be released to the release target. When there is no release target within the predetermined range from the mobile body, the smell releasing device is controlled to stop releasing the smell substance to the outside. When there is no release target within the predetermined range from the mobile body, it is not necessary to release the smell substance. Thus, by stopping the release of the smell substance to the outside, the consumption of the smell substance can be suppressed. Further, when there is no release target within the predetermined range from the mobile body, the release blocking device is controlled so that the release of the smell substance from the storage device to the communication portion is blocked. As a result, it is possible to stop the smell substance from being released to the outside by using the release blocking device. Further, when there is the release target within the predetermined range from the mobile body and the distance to the release target is long, the air flow of the blower is increased compared to when the distance is short. In this way, by increasing the air flow of the blower, the smell substance can reach the release target. As a result, the time during which the smell substance can be released can be increased, and the smell substance can reach the release target. Here, the “predetermined range” is a range that is predetermined as a range in which the smell substance is desired to reach. Examples of the “release target” include humans and non-human animals.

An overview of the control device of the present disclosure is that it is a control device that is installed in a mobile body together with a smell releasing device that releases to the outside, a smell substance that generates smell, in which when there is a release target to which the smell substance is released within a predetermined range from the mobile body, the control device controls the smell releasing device to release the smell substance to the outside, and when there is no release target within the predetermined range from the mobile body, the control device controls the smell releasing device to stop the smell substance from being released to the outside.

In the control device of the present disclosure, when there is a release target to which a smell substance is released within a predetermined range from the mobile body, the smell releasing device is controlled to release the smell substance to the outside. As a result, the smell substance can be released to the release target. When there is no release target within the predetermined range from the mobile body, the smell releasing device is controlled to stop releasing the smell substance to the outside. When there is no release target within the predetermined range from the mobile body, it is not necessary to release the smell substance. Thus, by stopping the release of the smell substance to the outside, the consumption of the smell substance can be suppressed. As a result, the time during which the smell substance can be released can be increased. Here, the “predetermined range” is a range that is predetermined as a range in which the smell substance is desired to reach. Examples of the “release target” include humans and non-human animals.

The overview of the program of the present disclosure is that it is a program that causes a computer to function as a control device installed in a mobile body together with a smell releasing device that releases to the outside, a smell substance that generates smell,

in which the program has a step of controlling the smell releasing device to release the smell substance to the outside when there is a release target to which the smell substance is released within a predetermined range from the mobile body, and controlling the smell releasing device to stop the smell substance from being released to the outside when there is no release target within the predetermined range from the mobile body.

In the program of the present disclosure, when there is a release target to which a smell substance is released within a predetermined range from the mobile body, the smell releasing device is controlled to release the smell substance to the outside. As a result, the smell substance can be released to the release target. When there is no release target within the predetermined range from the mobile body, the smell releasing device is controlled to stop releasing the smell substance to the outside. When there is no release target within the predetermined range from the mobile body, it is not necessary to release the smell substance. Thus, by stopping the release of the smell substance to the outside, the consumption of the smell substance can be suppressed. As a result, the time during which the smell substance can be released can be increased. Here, the “predetermined range” is a range that is predetermined as a range in which the smell substance is desired to reach. Examples of the “release target” include humans and non-human animals.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a configuration diagram showing an outline of a configuration of a vehicle 20 serving as a mobile body according to an embodiment of the present disclosure;

FIG. 2 is a configuration diagram showing an example of the configuration of a smell releasing device 22;

FIG. 3 is a schematic view schematically showing how a smell substance is released from a release port 220;

FIG. 4 is a flowchart showing an example of a smell release processing routine executed by an air conditioner ECU 40;

FIG. 5 is an explanatory diagram showing an example of the relationship between a setting distance D* and air blowing level AF;

FIG. 6 is a schematic view schematically showing how a smell substance is released from the release port 220 when a predetermined structure S is on one surface side of a vehicle; and

FIG. 7 is a flowchart showing an example of a smell release processing routine of a modified example executed by the air conditioner ECU 40.

### DETAILED DESCRIPTION OF EMBODIMENTS

Next, modes for carrying out the disclosure will be described using an embodiment.

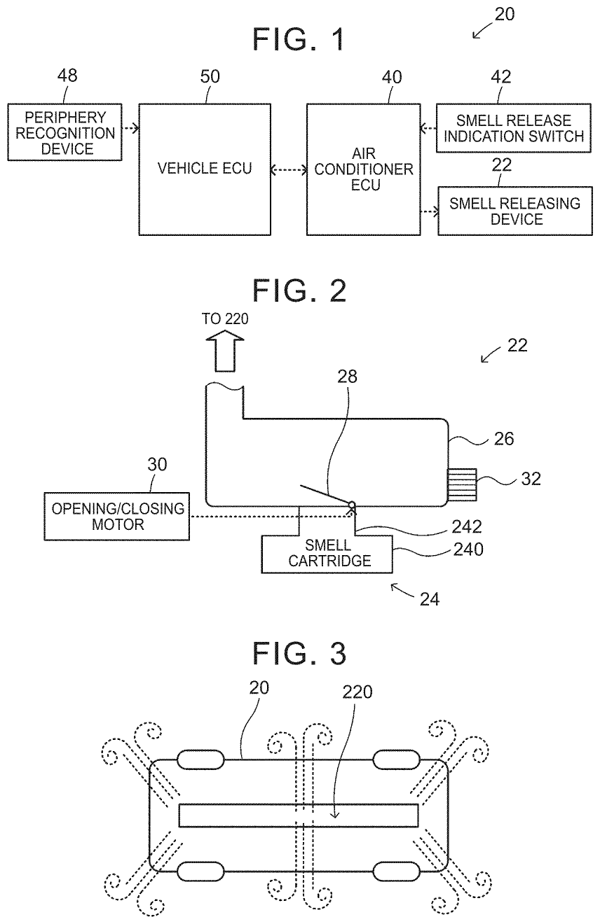

FIG. 1 is a configuration diagram showing an outline of a configuration of a vehicle 20 serving as a mobile body according to an embodiment of the present disclosure. The vehicle 20 is configured as an automobile used as a mobile store that sells food such as bread and cake, and has a smell releasing device 22, an air conditioner electronic control unit (hereinafter referred to as an “air conditioner ECU”) 40, and a vehicle electronic control unit (hereinafter referred to as a “vehicle ECU”) 50 in addition to a driving power source (an engine, a motor, etc.) not shown.

FIG. 2 is a configuration diagram showing an example of the configuration of the smell releasing device 22. FIG. 3 is a schematic view schematically showing how a smell substance is released from a release port 220. In FIG. 3, a broken line shows an example of the flow of the smell substance. The smell releasing device 22 is disposed near an air conditioner (not shown), and includes a smell cartridge 24, a communication portion 26, an opening/closing door 28, an opening/closing motor 30, and a blower (blower motor) 32.

The smell cartridge 24 has a storage portion 240 for storing a solution containing the smell substance that generates the smell of food to be sold or porous silica beads impregnated with such a solution, and an opening portion 242 with an open top portion in FIG. 3. The opening portion 242 is fitted in an opening formed in the communication portion 26 so that the opening portion 242 is detachable. The communication portion 26 allows communication between the smell cartridge 24 and the release port 220 formed on a ceiling of the vehicle 20. The opening/closing door 28 is attached to the opening portion 242. The opening/closing door 28 opens/closes the opening portion 242 to release and block the release of the smell substance from the storage portion 240. The opening/closing door 28 is driven by the opening/closing motor 30. The blower 32 is disposed on an upstream side of the smell cartridge 24 of the communication portion 26. The blower 32 takes in air and sends air from the upstream side of the smell cartridge 24 of the communication portion 26 toward a downstream side of the smell cartridge 24. The blower 32 and the opening/closing motor 30 are controlled by the air conditioner ECU 40. In the smell releasing device 22 configured in this way, when the opening/closing door 28 is open, the smell substance volatilized from the storage portion 240 is released to the communication portion 26 through the opening portion 242. The smell substance released into the communication portion 26 flows to the downstream side by the air sent by the blower 32, and is released to the outside from the release port 220. When the opening/closing door 28 is closed, the smell substance volatilized from the storage portion 240 is blocked from being released into the communication portion 26. As a result, the release of the smell substance from the release port 220 to the outside is stopped.

Although not shown, the air conditioner ECU 40 is configured as a microprocessor centered around a central processing unit (CPU), and includes a ready-only memory (ROM) for storing a processing program, a random access memory (RAM) for temporarily storing data, an input-output port, and a communication port, in addition to the CPU. The room temperature from a temperature sensor (not shown) that detects the temperature in a passenger compartment, a smell release signal Ssm from a smell releasing instruction switch 42, and the like are input to the air conditioner ECU 40. Various control signals are output from the air conditioner ECU 40 via an output port. Examples of the signals output from the air conditioner ECU 40 are a drive signal to a compressor (not shown), a drive signal to a blower (blower motor) for an air conditioner, a drive signal to the opening/closing motor 30, a drive signal to a blower 32, and the like. The air conditioner ECU 40 exchanges various data with the vehicle ECU 50 by communication.

A periphery recognition device 48 is configured of a camera, a millimeter wave radar, a quasi-millimeter wave radar, an infrared laser radar, a sonar, and the like. The periphery recognition device 48 recognizes positions and distances of people, other vehicles, and various structures around the vehicle 20. Based on a recognition result regarding the people around the vehicle 20, the periphery recognition device 48 detects a nearby people number Nh, which is the number of people in a predetermined range Rref from the vehicle 20, and a distance D(k) (k=1 to Nh) from the vehicle 20 to each person in the predetermined range Rref. Here, the “predetermined range Rref” is a range that is predetermined as a range in which the smell from the smell releasing device 22 is desired to reach, and is within a circle having a radius of 10 m around the vehicle 20, for example.

Although not shown, the vehicle ECU 50 is configured as a microprocessor centered on a CPU, and includes a ROM for storing a processing program, a RAM for temporarily storing data, an input-output port, and a communication port, in addition to the CPU. Signals from various sensors are input to the vehicle ECU 50. Examples of the signals input to the vehicle ECU 50 include detection signals from various sensors for detecting the state of a power source for traveling (not shown). Further, the nearby people number Nh and the distance D(k) (k=1 to Nh) from the periphery recognition device 48 are examples of the detection signals. Various control signals are output from the vehicle ECU 50 via the output port. Examples of the signal output from the vehicle ECU 50 include a control signal for controlling a power source for traveling and an occupant. The vehicle ECU 50 executes various calculations for driving the vehicle 20.

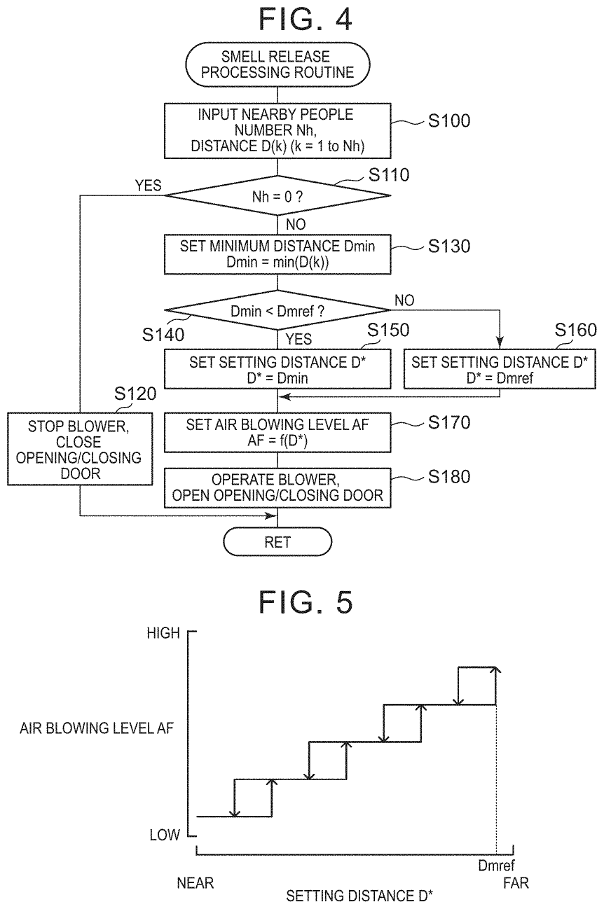

Next, the operation of the vehicle 20 configured in this way, particularly the operation when the smell substance is released from the smell releasing device 22 will be described. FIG. 4 is a flowchart showing an example of a smell release processing routine executed by the air conditioner ECU 40. This routine is performed repeatedly at every predetermined time (for example, several msec), from when the smell releasing instruction switch 42 is turned on when the vehicle 20 is parked until the movement of the vehicle 20 is started or until the smell releasing instruction switch 42 is turned off.

When this routine is executed, the CPU of the air conditioner ECU 40 executes a process of inputting the nearby people number Nh and the distance D(k) (k=1 to Nh) (step S100). The nearby people number Nh and the distance D(k) (k=1 to Nh) are input by inputting the nearby people number Nh and the distance D(k) (k=1 to Nh) recognized by the periphery recognition device 48 by communication via the vehicle ECU 50.

Next, it is determined whether the nearby people number Nh is 0 (step S110). When the nearby people number Nh is 0, that is, when there are no people within the predetermined range Rref from the vehicle 20, it is determined that it is not necessary to release the smell substance to the outside, and the blower 32 is stopped and the opening/closing motor 30 is controlled so that the opening/closing door 28 is closed (step S120), and this routine is finished. By closing the opening/closing door 28, the release of the smell substance from the smell cartridge 24 of the smell releasing device 22 to the outside is stopped. As described above, when there are no people in the predetermined range Rref from the vehicle 20, the smell substance is not released from the smell releasing device 22 and thus, the consumption of the smell substance can be suppressed. This makes it possible to increase the time during which the smell substance can be released.

When the nearby people number Nh is not 0 in step S110, that is, when there are people within the predetermined range Rref from the vehicle 20, the shortest distance among the distances D(k) (k=1 to Nh) is set as a minimum distance Dmin (step S130). Here, when the nearby people number Nh is 1, the corresponding distance D(1) is set as the minimum distance Dmin.

Subsequently, it is determined whether the minimum distance Dmin is less than a threshold value Dmref (step S140). The threshold value Dmref is a threshold value for determining whether there is a person in the immediate vicinity of the vehicle 20, and is set to 4 m, 5 m, 6 m, or the like, for example.

When the minimum distance Dmin is less than the threshold value Dmref in step S140, it is determined that there is a person in the immediate vicinity of the vehicle 20, and the minimum distance Dmin is set to a setting distance D* (step S150). Then, the setting distance D* is used to set the air blowing level AF of the blower 32 (step S170). The blower 32 is then controlled so that the blower can send air at the set air blowing level AF, and the opening/closing motor 30 is controlled so that the opening/closing door 28 is opened (step S180) and thus, this routine is finished.

FIG. 5 is an explanatory diagram showing an example of the relationship between the setting distance D* and the air blowing level AF. The air blowing level AF corresponds to the air flow (wind speed) that a person at the setting distance D* from the vehicle 20 does not feel uncomfortable. The air blowing level AF is set so that the air flow of the blower 32 increases when the air blowing level AF is high than when it is low, and as shown in the figure, it is set to increase in steps as the setting distance D* increases. Further, hysteresis is provided so that the air blowing level AF with respect to the setting distance D* becomes a different value when the setting distance D* increases and when the setting distance D* decreases from the previous value. Due to such hysteresis, the air blowing level AF is set so as not to increase or decrease frequently as the setting distance D* increases or decreases. Since the minimum distance Dmin is set to the setting distance D* in step S150, the blower 32 sends air at an air flow (wind speed) that does not make the person closest to the vehicle 20 uncomfortable. Since the air is sent in this way, it is possible to suppress a strong wind containing the smell substance from hitting the person in the immediate vicinity of the vehicle 20. Further, since the smell substance reaches a certain distance farther than the minimum distance Dmin due to the air being sent in this way, the smell of the food sold in the vehicle 20 can be transmitted to the people around the vehicle 20, and the appeal effect can be enhanced.

When the minimum distance Dmin is equal to or greater than the threshold Dmref in step S140, it is determined that there are no people in the immediate vicinity of the vehicle 20, and the threshold Dmref is set to the setting distance D* (step S160). Then, the setting distance D* is used to set the air blowing level AF of the blower 32 (step S170). The blower 32 is then controlled so that air is sent at the set air blowing level AF, and the opening/closing motor 30 is controlled so that the opening/closing door 28 is opened (step S180) and thus, the routine is finished. Since the threshold value Dmref is set to the setting distance D* in step S160, the blower 32 sends air at an air flow (wind speed) that does not make the person near the threshold value Dmref feel uncomfortable. Since the smell substance reaches a certain distance from the threshold value Dmref by the air being sent in this way, the smell of the food sold in the vehicle 20 can be transmitted to the people around the vehicle 20, and the appeal effect can be enhanced.

According to the vehicle 20 of the embodiment described above, when the nearby people number Nh is 0, the smell releasing device 22 is controlled so that the release of the smell substance is stopped and thus, the time during which the smell substance can be released can be increased.

Further, when the value of the nearby people number Nh is not 0, the smell releasing device 22 is controlled so that the smell substance is released and thus, the appealing effect can be enhanced.

Furthermore, when the value of the nearby people number Nh is not 0, the air blowing level AF is set so as to be increased in steps as the setting distance D* is increased and the blower 32 is controlled by the air blowing level AF. Thus, the smell substance can be more surely reach the people.

When the value of the nearby people number Nh is not 0 and the minimum distance Dmin is smaller than the threshold value Dmref, the minimum distance Dmin is set to the setting distance D*, and the blower 32 is controlled so that the air is sent at the air blowing level AF based on the setting distance D*. Thus, it is possible to suppress the inconvenience of hitting a person in the immediate vicinity of the vehicle 20 with a relatively large air flow.

In the vehicle 20 of the embodiment, when the minimum distance Dmin is equal to or greater than the threshold Dmref in steps S140 and S160, the threshold Dmref is set as the setting distance D*. However, when the minimum distance Dmin is equal to or greater than the threshold value Dmref, the minimum distance Dmin may be set as the setting distance D*. In this way, the wind containing the smell substance that hits the person closer to the vehicle 20 can be adjusted to an appropriate air flow.

In the vehicle 20 of the embodiment, when the value of the nearby people number Nh is not 0 in step S110, the blower 32 is controlled so as to execute step S130 and the subsequent steps to drive the blower 32 at the air blowing level AF according to the setting distance D*. However, when the value of the nearby people number Nh is not 0 in step S110, the blower 32 may be controlled at a predetermined air blowing level AF without executing step S130 and the subsequent steps.

In the vehicle 20 of the embodiment, the air blowing level AF is set in step S170 so as to be increased in steps as the setting distance D* is increased. However, the air blowing level AF may be set to be monotonically increased with respect to the setting distance D*.

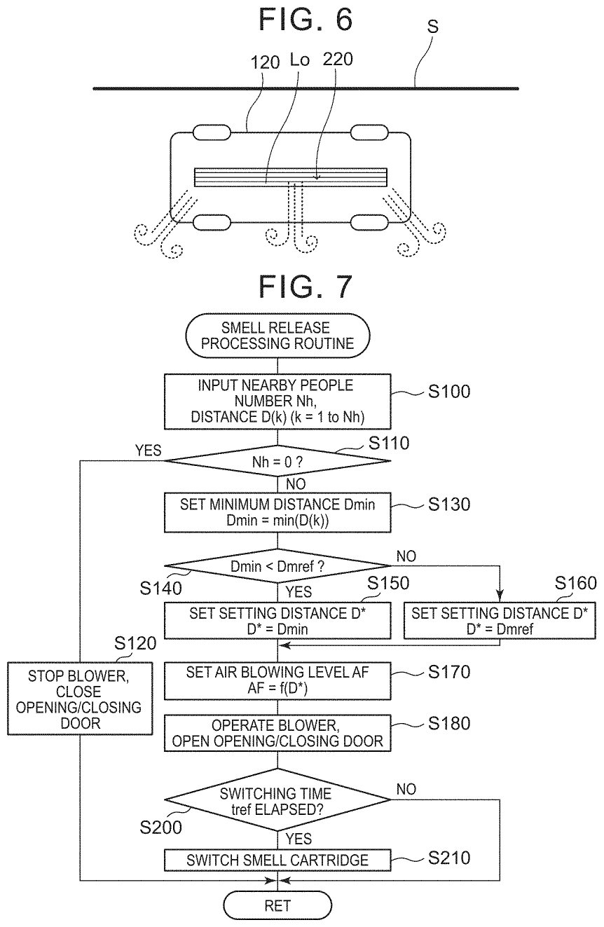

In the vehicle 20 of the embodiment, a smell substance is released from the release port 220 to the periphery of the vehicle 20. However, a movable louver Lo may be provided near the release port 220, and the releasing direction of the smell substance may be adjusted by changing the angle of the louver Lo. In this case, when there is a predetermined structure S on one surface side of the vehicle to which the release of smells should be avoided such as a wall or a private house, the angle of the louver Lo may be adjusted so as not to release the smell substance in the direction of the predetermined structure S. FIG. 6 is a schematic view that schematically shows how a smell substance is released from a release port 220 when the predetermined structure S is on one surface side of the vehicle. By adjusting the angle of the louver Lo in this way, it is possible to suppress the release of the smell substance to the predetermined structure S and release the smell substance in a direction different from that of the predetermined structure S.

In the vehicle 20 of the embodiment, the smell releasing device 22 includes one smell cartridge 24. However, the smell releasing device 22 may include a plurality of the smell cartridges 24 that store different smell substances. Then, the smell cartridges 24 may be attached to the communication portion 26, and each opening/closing door 28 may be controlled to switch the smell cartridge 24 in communication with the communication portion 26. In this case, instead of the smell release processing routine illustrated in FIG. 4, the smell release processing routine of a modified example illustrated in FIG. 7 may be executed. The smell release processing routine of the modified example is the same as the smell release processing routine of FIG. 4 except for steps S200 and S210 that are executed after step S180. Thus, in the description of the smell release processing routine of the modified example, the same processing as that of the smell release processing routine of FIG. 4 has the same reference numerals, and the description thereof will be omitted.

In the smell release processing routine of the modified example, after controlling the blower 32 in step S180, it is determined whether a predetermined time (switching time) tref has elapsed since the smell cartridge 24 was previously switched (step S200). The predetermined time (switching time) tref is the time predetermined as the time for switching the type of the smell substance to be released and is set to 30 minutes, 1 hour, or 1 hour and a half, for example. When the predetermined time (switching time) tref has not elapsed since the smell cartridge 24 has been previously switched, the present routine is finished without the smell substance that is released to the communication portion 26 being changed. When the predetermined time (switching time) tref has elapsed since the smell cartridge 24 has been previously switched, the opening/closing door 28 provided in the opening portion 242 of each smell cartridge 24 is controlled so that the smell substance released to the communication portion 26 is switched and then, the smell cartridge 24 that releases the smell to the communication portion 26 is switched (step S210). Thus, the smell release processing routine of the modified example is finished. Since the smell substance released to the communication portion 26 is switched every predetermined time (switching time) tref, the smell substance diffused to the outside from the release port 220 can be switched. As a result, a different smell can be transmitted to the people around the vehicle 20 every predetermined time (switching time) tref.

In steps S200 and S210 of the smell release processing routine in FIG. 7, when the predetermined time (switching time) tref has elapsed since the smell cartridge 24 has been previously switched, the smell cartridge 24 in communication with the communication portion 26 is switched. However, the vehicle 20 may be provided with a changeover switch for instructing the changeover of the smell cartridge 24, and when the changeover switch instructs the changeover of the smell cartridge 24, the smell cartridge 24 in communication with the communication portion 26 may be switched.

In the vehicle 20 of the embodiment, the smell release processing routine of FIG. 4 is executed from when the smell releasing instruction switch 42 is turned on when the vehicle 20 is parked until the movement of the vehicle 20 is started or the smell releasing instruction switch 42 is turned off. However, the smell release processing routine of FIG. 4 may be executed from when the vehicle 20 is parked until the movement of the vehicle 20 is started, regardless of whether the smell releasing instruction switch 42 is turned on or off. Further, the smell releasing routine in FIG. 4 may be executed from when the smell releasing instruction switch 42 is turned on while the vehicle 20 is moving such as when the vehicle 20 is traveling at a low speed until the smell releasing instruction switch 42 is turned off.

In the vehicle 20 of the embodiment, the opening/closing door 28 is provided in the opening portion 242 of the smell cartridge 24, and the opening/closing door 28 is opened/closed to release the smell substance to the communication portion 26 and to stop the release. However, when the liquid smell substance is stored in the storage portion 240, a suction/spray device that sucks up the liquid smell substance of the storage portion 240 into the opening portion 242 and sprays the smell substance onto the communication portion 26 may be provided without the opening/closing door 28 and the opening/closing motor 30 being provided, and the release and the stopping of the release of the smell to the communication portion 26 may be performed by the operation and stopping of the suction/spray device.

In the vehicle 20 of the embodiment, the opening/closing door 28 is attached to the opening portion 242 of the smell cartridge 24 so that the opening/closing door 28 can be opened and closed. However, the opening/closing door 28 may be attached to the release port 220 so that the opening/closing door 28 can be opened and closed.

In the vehicle 20 of the embodiment, the smell cartridge 24 has the storage portion 240 for storing a solution containing the smell substance that generates the smell of food to be sold or porous silica beads impregnated with such a solution, and the opening portion 242 that is fitted to the communication portion 26 so as to be detachable. However, when the smell cartridge 24 is equipped with an electronic control unit configured as a microprocessor centered around a CPU and the smell cartridge 24 is mounted on the communication portion 26, the electronic control unit, the air conditioner ECU 40, and the vehicle ECU 50 may be able to communicate with each other, and the electronic control unit may execute the smell release processing routine of FIG. 4 instead of the air conditioner ECU 40.

In the vehicle 20 of the embodiment, the target for releasing the smell substance are people. However, non-human animals such as dogs and cats can also be mentioned as targets to which the smell substances are released.

The embodiment illustrates the case in which the present disclosure is applied to a vehicle used as a mobile store for selling food. However, the present disclosure is not limited to a vehicle used as a store, and may be applied to a vehicle for any purpose as long as it is a vehicle installed with a smell releasing device that releases a smell substance to the outside. In this case, the smell releasing device may release the smell substance corresponding to the application of the vehicle.

The embodiment illustrates the case in which the present disclosure is applied to a vehicle. However, the present disclosure may be applied to various mobile bodies different from a vehicle, such as an autonomous traveling robots and a movable construction machine. Further, the present disclosure may be in the form of a control device installed in such a mobile body, or may be in the form of a program for making the air conditioner ECU 40 and the vehicle ECU 50 function as a control device mounted on a mobile body.

The correspondence relationship between the main elements of the embodiment and the main elements of the disclosure described in the column of the summary of the disclosure will be described. In the embodiment, the smell releasing device 22 corresponds to the “smell releasing device”, and the air conditioner ECU 40, the periphery recognition device 48, and the vehicle ECU 50 correspond to the “control device”.

The correspondence between the main elements of the embodiment and the main elements of the disclosure described in the summary of the disclosure is an example for specifically describing a mode for carrying out the disclosure described in the summary of the disclosure. Therefore, the embodiment does not limit the elements of the disclosure described in the summary of the disclosure. That is, the interpretation of the disclosure described in the summary of the disclosure should be carried out based on the description in the summary of the disclosure, and the embodiment is merely a specific example of the disclosure described in the summary of the disclosure.

Although the mode for carrying out the disclosure has been described above with reference to the embodiment, the disclosure is not limited to the embodiment, and the disclosure may be carried out in various modes without departing from the gist of the disclosure.

The present disclosure can be used in the manufacturing industry of mobile bodies, smell cartridges, control devices, programs, and the like.

What is claimed is:

1. A mobile body comprising:
a smell releasing device that releases to the outside, a smell substance that generates smell; and
a control device that controls the smell releasing device,
wherein when there is a release target to which the smell substance is released within a predetermined range from the mobile body, the control device controls the smell releasing device to release the smell substance to the outside, and when there is no release target within the predetermined range from the mobile body, the control device controls the smell releasing device to stop the smell substance from being released to the outside.

2. The mobile body according to claim 1, wherein when there is the release target within the predetermined range from the mobile body and a distance from the mobile body to the release target is long, the control device controls the smell releasing device to increase a reaching distance of the smell substance compared to when the distance is short.

3. The mobile body according to claim 2,
wherein the smell releasing device has a storage device that stores the smell substance, a communication portion that allows communication between the storage device and the outside, a release blocking device that is able to block the smell substance from being released from the storage device to the communication portion, and a blower that takes in air and sends air to the communication portion,
wherein when there is no release target within the predetermined range from the mobile body, the control device controls the release blocking device to block the smell substance from being released from the storage device to the communication portion, and when there is the release target within the predetermined range from the mobile body and the distance to the release target is long, the control device increases an air flow of the blower compared to when the distance is short.

4. The mobile body according to claim 3, wherein when there is a plurality of release targets within the predetermined range from the mobile body and a distance from the mobile body to the release target closest to the mobile body among the plurality of release targets is long, the control device increases the air flow of the blower compared to when the distance is short.

5. The mobile body according to claim 1,
wherein the mobile body includes a direction adjusting device that adjusts a releasing direction of the smell substance, and
wherein when there is, on one surface side of the mobile body, a predetermined object in which release of the smell substance should be avoided, the control device controls the direction adjusting device to suppress the smell substance from being released to the one surface side.

6. A smell cartridge used for the mobile body according to claim 3, the smell cartridge comprising:
a storage portion that functions as the storage device when used in the mobile body; and
a control unit that functions as the control device when used in the mobile body.

7. A control device that is installed in a mobile body together with a smell releasing device that releases to the outside, a smell substance that generates smell, wherein when there is a release target to which the smell substance is released within a predetermined range from the mobile body, the control device controls the smell releasing device to release the smell substance to the outside, and when there is no release target within the predetermined range from the mobile body, the control device controls the smell releasing device to stop the smell substance from being released to the outside.

8. A program that causes a computer to function as a control device installed in a mobile body together with a smell releasing device that releases to the outside, a smell substance that generates smell, wherein the program has a step of controlling the smell releasing device to release the smell substance to the outside when there is a release target to which the smell substance is released within a predetermined range from the mobile body, and controlling, the smell releasing device to stop the smell substance from being released to the outside when there is no release target within the predetermined range from the mobile body.
